(19) United States
(12) Patent Application Publication
(10) Pub. No.: US 2021/0148843 A1
(43) Pub. Date: May 20, 2021

(54) GAS SENSOR

(71) Applicant: Nuvoton Technology Corporation, Hsinchu Science Park (TW)

(72) Inventors: Po-Kai HUANG, Hsinchu County (TW); Ming-Chih TSAI, Taichung City (TW); Chih-Hsuan CHIEN, Taoyuan City (TW)

(21) Appl. No.: 16/999,936

(22) Filed: Aug. 21, 2020

(30) Foreign Application Priority Data
Nov. 18, 2019 (TW) 108141733

(51) Int. Cl. G01N 27/12; G01N 33/00

(52) U.S. Cl. CPC G01N 27/127; G01N 33/0047

(57) ABSTRACT

A gas sensor is provided. The gas sensor includes a substrate, a plurality of electrodes formed on the substrate, and a metal layer formed on the substrate and the electrodes. The metal layer includes a plurality of first molecules doped with a plurality of second molecules. Each of the first molecules includes a metal particle and a plurality of carbon chains connected to a surface of the metal particle. Each of the second molecules includes a conjugated structure.

### CROSS-REFERENCE TO RELATED APPLICATIONS

The present application claims priority of Taiwan Application No. 108141733, filed on Nov. 18, 2019, which is incorporated by reference herein in its entirety.

### BACKGROUND

### Technical Field

The disclosure relates to a gas sensor, and more particularly to a gas sensor capable of effectively avoiding aggregation of nano-metal particles.

### Description of the Related Art

In general, gas sensors can be divided into six types, namely metal oxide type, conductive polymer type, optical catalyst type, quartz crystal microbalance type, surface acoustic wave type, and chemi-resistor type.

Nano-gold particles are often used as sensing materials in chemi-resistor type gas sensors. However, there are two problems with these materials. One is the low conductivity of the capping agent, which provides the stability of nano-gold particles. It causes the resistance of the resulting nano-gold thin film to be too high and difficult to control. The resistance often reaches tens to hundreds of Mega Q, which makes people face enormous difficulties in designing back-end signal processing circuits. The other relates to the service life of the device. Due to the characteristics of nano-gold particles, they will continue to aggregate over time, resulting in a continuous decrease in the resistance change rate during sensing, and eventually making the sensor unusable.

Therefore, it is desirable to develop a gas sensor that can effectively avoid the aggregation of nano-metal particles and improve the sensing performance.

### SUMMARY

In accordance with some embodiments of the present disclosure, a gas sensor is provided. The gas sensor includes a substrate, a plurality of electrodes formed on the substrate, and a metal layer formed on the substrate and the electrodes, wherein the metal layer includes a plurality of first molecules doped with a plurality of second molecules, wherein each of the first molecules includes a metal particle and a plurality of carbon chains connected to surfaces of the metal particle, and each of the second molecules includes a conjugated structure.

In some embodiments, the metal particle in the first molecules includes Au, Ag, Cu, Sn, Pd, Pt, Ni, Co, or Al. In some embodiments, a number of carbon atoms in the carbon chains in the first molecules are between 6 and 24. In some embodiments, the carbon chains in the first molecules are connected to the surface of the metal particle through an anchor unit. In some embodiments, the anchor unit includes an S atom, a P atom, or an N atom.

In some embodiments, the second molecules include a nitrogen-containing cyclic conjugated structure, a sulfur-containing cyclic conjugated structure, or a cyclic conjugated structure with double bonds. In some embodiments, the second molecules include

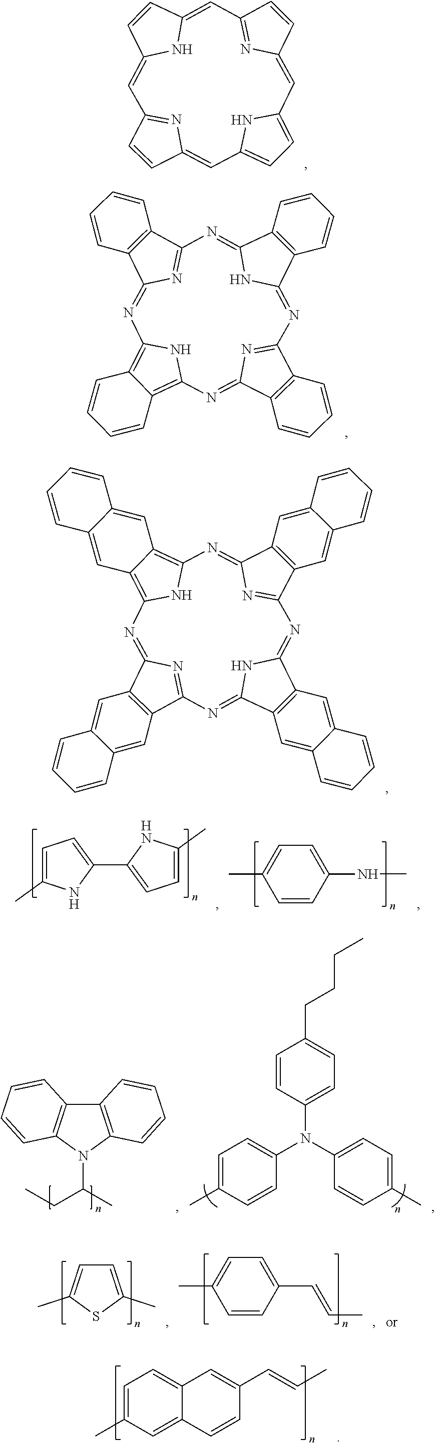

In some embodiments, the second molecules include a nitrogen-containing cyclic conjugated structure modified by functional groups, a sulfur-containing cyclic conjugated structure modified by functional groups, or a cyclic conjugated structure with double bonds modified by functional groups. In some embodiments, the second molecules include

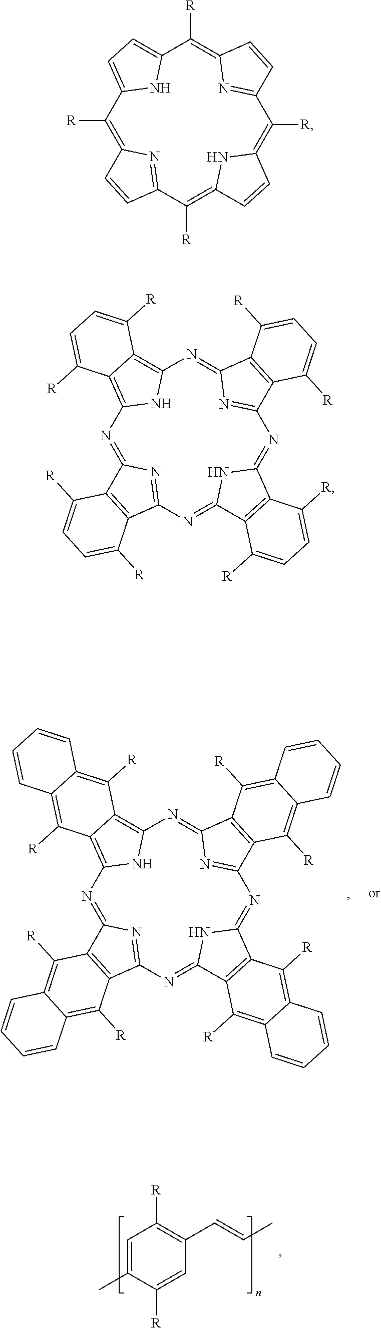

wherein R includes: heterocyclic compounds, —O— (CH₂)ₙH, —O—(CH₂CH₂O)ₙCH₃, —S(CH₂)ₙH, —O—(CH₂CH₂O)ₙSH,

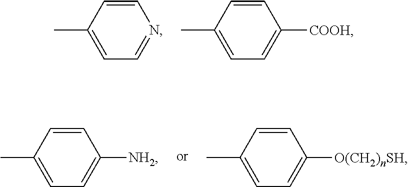

and n is between 0 and 24.

In some embodiments, the doping concentration ratio of the second molecules to the first molecules is between 1:2 and 1:100,000. In some embodiments, the doping concentration ratio of the second molecules to the first molecules is between 1:20 and 1:10,000.

In some embodiments, the first molecules are physically mixed with the second molecules. In some embodiments, the first molecules form covalent bonds with the second molecules. In some embodiments, the metal particle in the first molecules forms covalent bonds with the functional groups in the second molecules.

In some embodiments, target gases detectable by the gas sensor include volatile organic compounds gases. In some embodiments, target gases detectable by the gas sensor include amine gases, nitrogen oxide gases, or explosive gases.

In the present disclosure, an organic compound with a conjugated structure, such as porphyrin

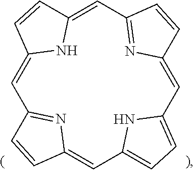 [SMILES: C1=Cc2cc3ccc(cc4nc(cc5ccc(cc1n2)[nH]5)C=C4)[nH]3]

phthalocyanine

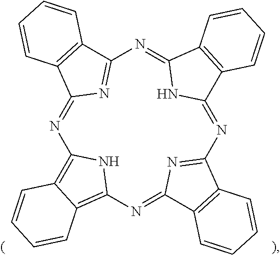 [SMILES: c1ccc2c(c1)-c1nc-2nc2[nH]c(nc3nc(nc4[nH]c(n1)c1ccccc41)-c1ccccc1-3)c1ccccc21]

or naphthalocyanine

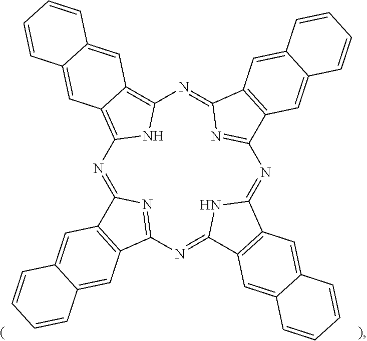 [SMILES: c1ccc2cc3c(cc2c1)-c1nc-3nc2[nH]c(nc3nc(nc4[nH]c(n1)c1cc5ccccc5cc41)-c1cc4ccccc4cc1-3)c1cc3ccccc3cc21]

is introduced into a nano-metal particle. The organic compound is further modified by functional groups to improve the bonding stability between the organic compound and the nano-metal particle. In the present disclosure, the doping concentration of the organic compound is adjusted and optimized to increase conductive path. The resistances of the gas sensors are precisely controlled to maintain the resistances within desired ranges, so as to effectively reduce the difficulty in integration among the gas sensors, semiconductor processes and signal processing circuits. Because the doped organic compound increases the distance between the nano-metal particles, it effectively inhibits the aggregation between the nano-metal particles, thereby increasing the service life of the device. Because the doped organic compound and functional groups on its side chains are non-polar, in addition to detecting polar gases, it becomes easier to catch non-polar gases. The doped organic compound can increase the change in resistance to achieve the effect of signal amplification, and further improve the sensitivity of the gas sensor.

### BRIEF DESCRIPTION OF THE DRAWINGS

FIG. 1 illustrates a cross-sectional view of a gas sensor according to an embodiment of the present disclosure;

FIG. 2 schematically illustrates a metal layer of a gas sensor according to an embodiment of the present disclosure;

FIG. 3 schematically illustrates a metal layer of a gas sensor according to an embodiment of the present disclosure;

FIG. 4 shows measurement results of physical properties of gas sensors according to an embodiment of the present disclosure;

FIG. 5 shows measurement results of physical properties of gas sensors according to an embodiment of the present disclosure; and

FIG. 6 shows measurement results of physical properties of gas sensors according to an embodiment of the present disclosure.

### DETAILED DESCRIPTION

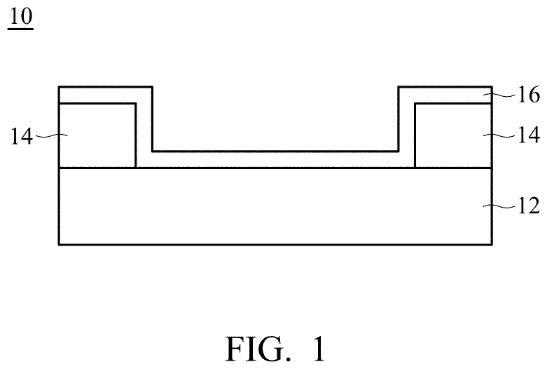

Referring to FIG. 1, a gas sensor 10 is provided in accordance with an embodiment of the present disclosure. FIG. 1 illustrates the cross-sectional view of the gas sensor 10.

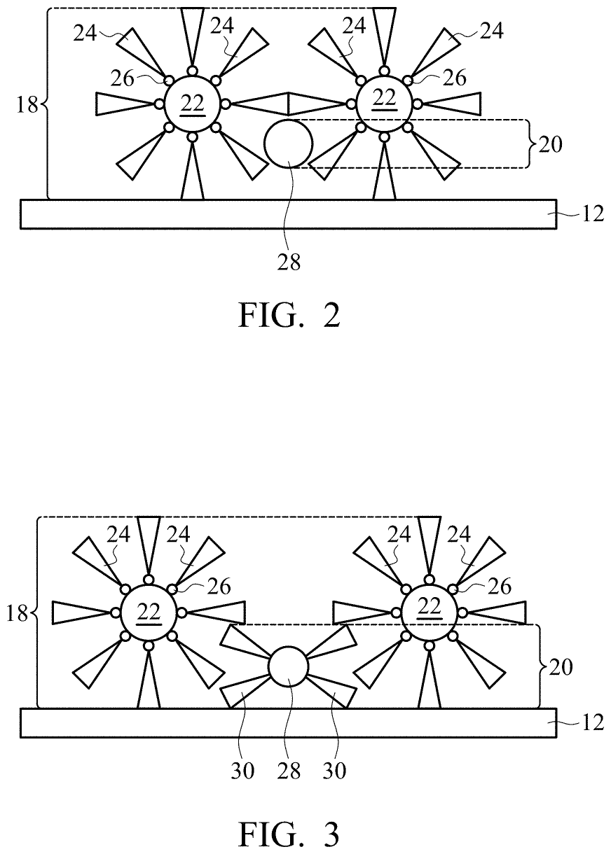

In FIG. 1, the gas sensor 10 includes a substrate 12, a plurality of electrodes 14, and a metal layer 16. The electrodes 14 are formed on the substrate 12. The metal layer 16 is formed on the substrate 12 and the electrodes 14. For example, the metal layer 16 is globally formed on the substrate 12 and the electrodes 14. In some embodiments, the substrate 12 may include Si, metal oxide, or other suitable substrate materials. In some embodiments, the electrodes 14 may include Au, Ag, Cu, or other suitable electrode materials. FIGS. 2 and 3 illustrate various configurations in the metal layer 16.

As shown in FIG. 2, in some embodiments, the metal layer 16 includes a plurality of first molecules 18 doped with a plurality of second molecules 20. Each of the first molecules 18 includes a metal particle 22 and a plurality of carbon chains 24 connected to the surface of the metal particle 22. Each of the second molecules 20 includes a core structure 28.

In some embodiments, the metal particle 22 in the first molecules 18 may include Au, Ag, Cu, Sn, Pd, Pt, Ni, Co, or Al. In some embodiments, a number of carbon atoms in the carbon chains 24 in the first molecules 18 are between 6 and 24. In some embodiments, a number of carbon atoms in the carbon chains 24 in the first molecules 18 are between 8 and 20. In some embodiments, the carbon chains 24 in the first molecules 18 are connected to the surface of the metal particle 22 through an anchor unit 26. In some embodiments, the anchor unit 26 may include an S atom, a P atom, or an N atom.

In some embodiments, the core structure 28 of the second molecules 20 may include a nitrogen-containing cyclic conjugated structure, a sulfur-containing cyclic conjugated structure, or a cyclic conjugated structure with double bonds. In some embodiments, the core structure 28 of the second molecules 20 may include

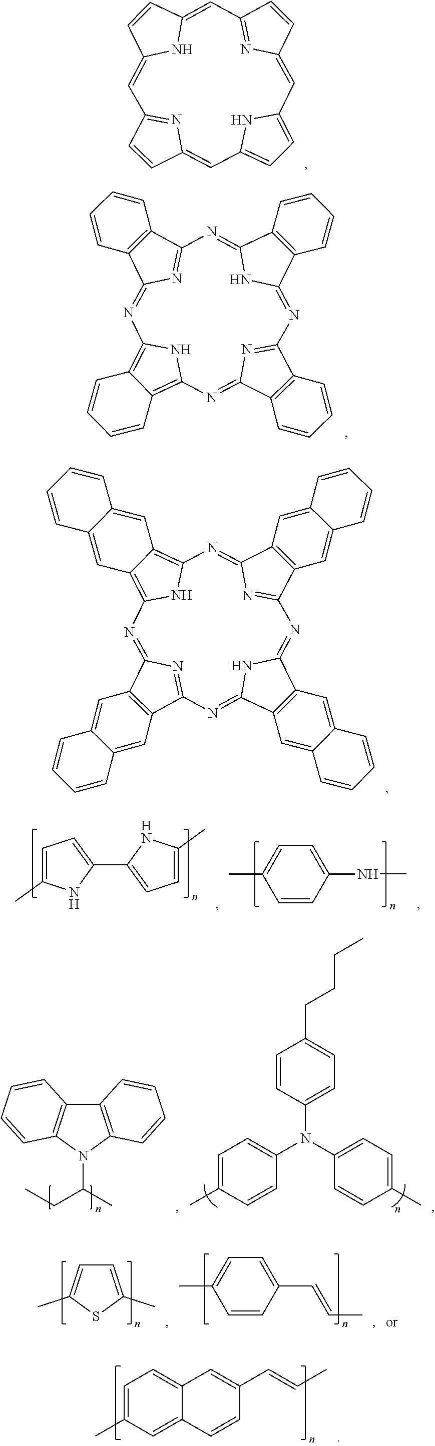

In some embodiments, a doping concentration ratio of the second molecules 20 to the first molecules 18 is between 1:2 and 1:100,000. In some embodiments, a doping concentration ratio of the second molecules 20 to the first molecules 18 is between 1:20 and 1:10,000.

In some embodiments, the first molecules 18 are physically mixed with the second molecules 20. In other words, the first molecules 18 do not form covalent bonds with the second molecules 20.

As shown in FIG. 3, in some embodiments, the metal layer 16 includes a plurality of the first molecules 18 doped with a plurality of the second molecules 20.

Each of the first molecules 18 includes the metal particle 22 and a plurality of the carbon chains 24 connected to the surface of the metal particle 22. Each of the second molecules 20 includes a core structure 28 and a plurality of functional groups 30 connected to the surface of the core structure 28.

In some embodiments, the metal particle 22 in the first molecules 18 may include Au, Ag, Cu, Sn, Pd, Pt, Ni, Co, or Al. In some embodiments, a number of carbon atoms in the carbon chains 24 in the first molecules 18 are between 6 and 24. In some embodiments, a number of carbon atoms in the carbon chains 24 in the first molecules 18 are between 8 and 20. In some embodiments, the carbon chains 24 in the first molecules 18 are connected to the surface of the metal particle 22 through the anchor unit 26. In some embodiments, the anchor unit 26 may include an S atom, a P atom, or an N atom.

In some embodiments, the second molecules 20 may include a nitrogen-containing cyclic conjugated structure modified by functional groups 30, a sulfur-containing cyclic conjugated structure modified by functional groups 30, or a cyclic conjugated structure with double bonds modified by functional groups 30. In some embodiments, the second molecules may include

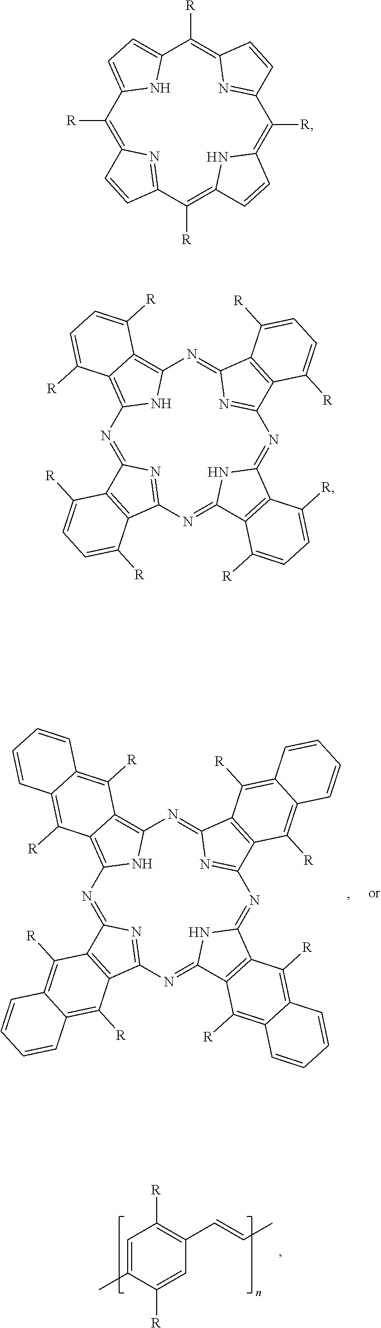

wherein R includes: heterocyclic compounds, —O—(CH₂)ₙH, —O—(CH₂CH₂O)ₙCH₃, —S(CH₂)ₙH, —O—(CH₂CH₂O)ₙSH,

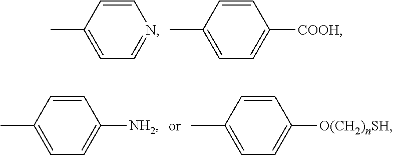

and n is between 0 and 24.

In some embodiments, a doping concentration ratio of the second molecules 20 to the first molecules 18 is between 1:2 and 1:100,000. In some embodiments, a doping concentration ratio of the second molecules 20 to the first molecules 18 is between 1:20 and 1:10,000.

In some embodiments, the first molecules 18 are physically mixed with the second molecules 20. In other words, the first molecules 18 do not form covalent bonds with the second molecules 20. In some embodiments, the first molecules 18 may form covalent bonds with the second molecules 20, for example, the metal particle 22 in the first molecules 18 forms covalent bonds with the functional groups 30 in the second molecules 20.

In the present disclosure, since the nano-metal particle in the metal layer 16 are soluble in various organic solvents, the nano-metal particle thin film may be deposited by, for example, drop coating or spraying. In some embodiments, the nano-metal particle thin film (e.g. the metal layer 16) may also be deposited by, for example, ink-jet printing (IJP), micro-contact printing, a glue dispenser, or photolithography.

In some embodiments, target gases detectable by the gas sensor 10 may include volatile organic compounds (VOCs) such as ethanol, toluene, butanol, or octane. In some embodiments, target gases detectable by the gas sensor 10 may include amine gases, nitrogen oxide gases, or explosive gases.

The sensing principle of the gas sensor of the present disclosure is that when the gas sensor is in contact with organic gas molecules, physical adsorption occurs between the gas molecules and the nano-metal particle. The diffusion of gas molecules into the gap between the metal particle increases the distance between two metal particles, which increases the path of electron jumping and tunneling and results in a decrease in conductivity and an increase in resistance. Due to the different interactions between various gas molecules and the nano-metal particle, the nano-metal particle has different physical adsorption capability to various volatile organic compounds gases. The degree of change in the distance between the metal particles caused by the adsorption of organic molecules is also different, so the gas sensor has different sensitivity to different gases. In addition, the nano-metal particle is selected as the sensing materials because they can be used at normal temperature and pressure, and they may react to most volatile organic compounds gases.

In the present disclosure, an organic compound with a conjugated structure, such as porphyrin

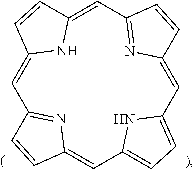 [SMILES: C1=Cc2cc3ccc(cc4nc(cc5ccc(cc1n2)[nH]5)C=C4)[nH]3]

phthalocyanine

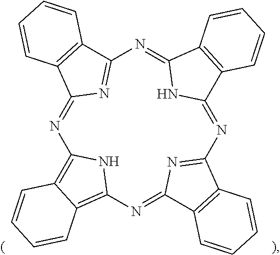 [SMILES: c1ccc2c(c1)-c1nc-2nc2[nH]c(nc3nc(nc4[nH]c(n1)c1ccccc41)-c1ccccc1-3)c1ccccc21]

or naphthalocyanine

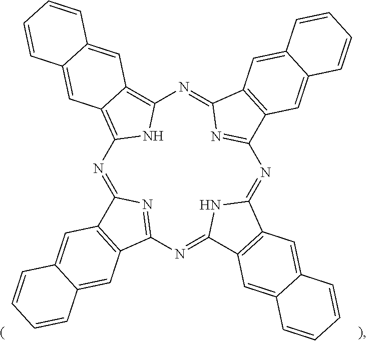 [SMILES: c1ccc2cc3c(cc2c1)-c1nc-3nc2[nH]c(nc3nc(nc4[nH]c(n1)c1cc5ccccc5cc41)-c1cc4ccccc4cc1-3)c1cc3ccccc3cc21]

is introduced into the nano-metal particle. The organic compound is further modified by functional groups to improve the bonding stability between the organic compound and the nano-metal particle. In the present disclosure, the doping concentration of the organic compound is adjusted and optimized to increase conductive paths. The resistances of the gas sensors are precisely controlled to maintain the resistances within desired ranges, so as to effectively reduce the difficulty in integration among the gas sensors, semiconductor processes and signal processing circuits. Because the doped organic compound increases the distance between the nano-metal particles, it effectively inhibits the aggregation between the nano-metal particles, thereby increasing the service life of the device. Because the doped organic compound and functional groups on its side chains are non-polar, in addition to detecting polar gases, it becomes easier to catch non-polar gases. The doped organic compound can increase the change of resistance to achieve the effect of signal amplification, and further improve the sensitivity of the gas sensor.

### Example 1

Measurement of Baseline Resistances of Gas Sensors

Example 1 illustrates the influence of the metal layer doped with conjugated molecules in the gas sensor on the baseline resistance of the gas sensor. First, Sensor C, Sensor I, Sensor II, Sensor III, and Sensor IV were provided. In Example 1, the metal layers in the gas sensors described above were mainly formed of nano-gold particles with octyl groups attached to the surface. The metal layer of Sensor C was not doped with conjugated molecules. The metal layers of Sensors I to IV were doped with conjugated molecules

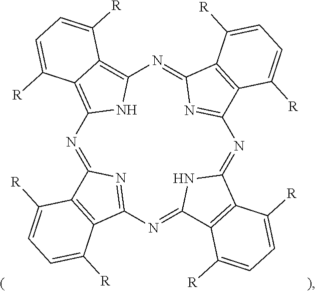

where R was —O—(CH₂)₃CH₃. The doping concentration ratios were 1:20 (Sensor I), 1:100 (Sensor II), 1:2,000 (Sensor III), and 1:10,000 (Sensor IV). The baseline resistances of the gas sensors were measured, and the measurement results are shown in Table 1.

TABLE 1

| Gas Sensor | Doping Concentration Ratio | Baseline Resistance | |
|---|---|---|---|
| Sensor C | 0 | ~500 | MΩ |
| Sensor I | 1:20 | 11.3 ± 1.1 | MΩ |
| Sensor II | 1:100 | 3.01 ± 0.14 | MΩ |
| Sensor III | 1:2,000 | 0.72 ± 0.02 | MΩ |
| Sensor IV | 1:10,000 | 0.28 ± 0.01 | MΩ |

Referring to Table 1, the baseline resistances of Sensors I to IV whose metal layers were doped with conjugated molecules can be precisely controlled. The baseline resistances of Sensors I to IV can be controlled within the desired values without generating excessive resistance variability by adjusting and optimizing the doping concentration of the conjugated molecules.

### Example 2

Measurement of Service Lives of Gas Sensors

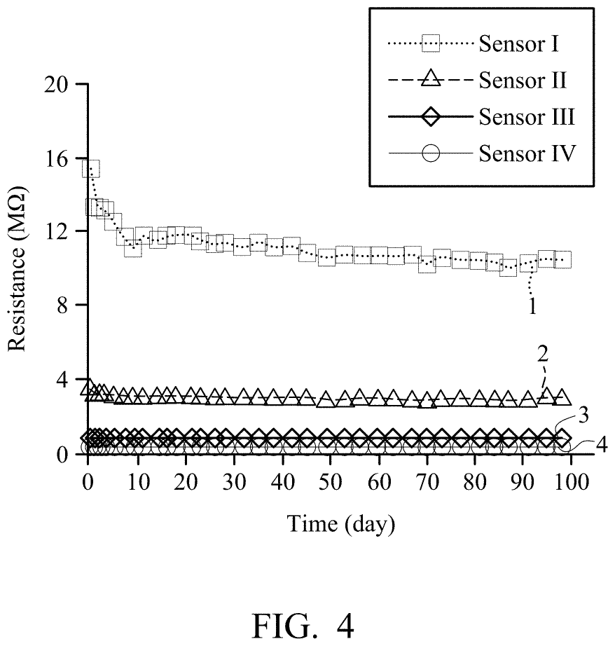

Example 2 illustrates the influence of the metal layers doped with conjugated molecules in the gas sensors on the service lives of the gas sensors. First, Sensor I, Sensor II, Sensor III, and Sensor IV were provided. In Example 2, the metal layers in the gas sensors described above were mainly formed of nano-gold particles with octyl groups attached to the surface. The metal layers of Sensors I to IV were doped with conjugated molecules

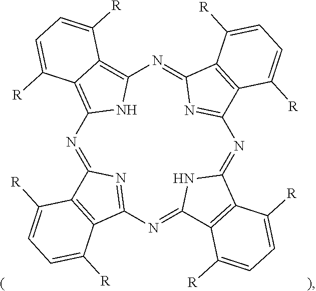

where R was —O—(CH₂)₃CH₃. The doping concentration ratios were 1:20 (Sensor I), 1:100 (Sensor II), 1:2,000 (Sensor III), and 1:10,000 (Sensor IV). The service lives of the gas sensors were measured, and the measurement results are shown in FIG. 4.

In FIG. 4, Curve 1 shows the change in the resistance of Sensor I over time. Curve 2 shows the change in the resistance of Sensor II over time. Curve 3 shows the change in the resistance of Sensor III over time. Curve 4 shows the change in the resistance of Sensor IV over time. As shown in FIG. 4, the functions of Sensors I to IV were maintained for more than several months (the resistances of the sensors changed very little over time) and were not affected by the environment and humidity. Because the conjugated molecules doped into the metal layers in Sensors I to IV increased the distances between nano-gold particles, they effectively inhibited the aggregation of nano-gold particles, thereby increasing the service lives of the gas sensors.

### Example 3

Measurement of Sensitivities of Gas Sensors

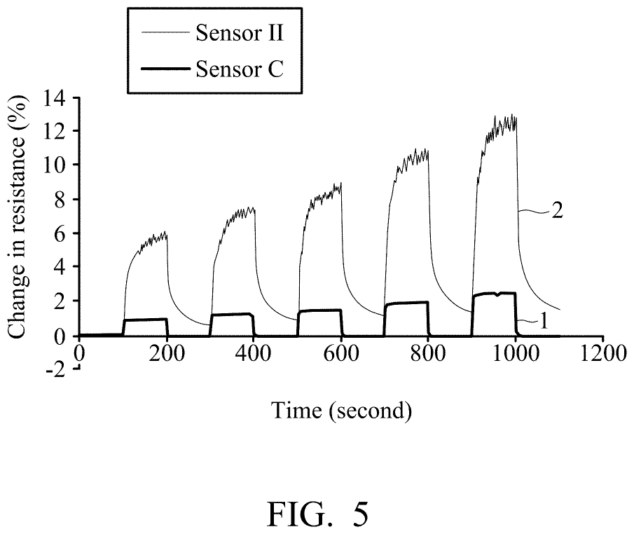

Example 3 illustrates the influence of the metal layers doped with conjugated molecules in the gas sensors on the sensitivities of the gas sensors. First, Sensor C and Sensor II were provided. In Example 3, the metal layers in the gas sensors described above were mainly formed of nano-gold particles with octyl groups attached to the surface. The metal layer of Sensor C was not doped with conjugated molecules. The metal layer of Sensor II was doped with conjugated molecules

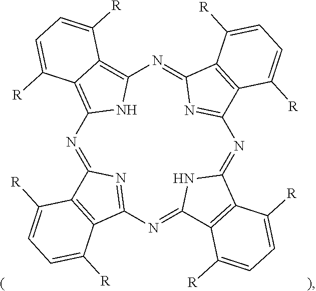

where R was —O—(CH₂)₃CH₃. The doping concentration ratio was 1:100. A target gas (e.g. toluene) with a concentration between 400 ppm and 1,000 ppm was then introduced into the gas sensors and the sensitivities of gas sensors were measured. 400, 500, 600, 800, and 1,000 ppm of toluene gases were introduced at 100, 300, 500, 700, and 900 seconds, respectively. The measurement results are shown in FIG. 5.

In FIG. 5, Curve 1 shows the change in the resistance of Sensor C after contacting with the target gas, and Curve 2 shows the change in the resistance of Sensor II after contacting with the target gas. As shown in FIG. 5, no matter what concentration of toluene gas was introduced and no matter what time the toluene gas was introduced, the change in the resistance of sensor C after contacting with the target gas was very small. In contrast, no matter what concentration of toluene gas was introduced and no matter what time the toluene gas was introduced, the change in the resistance of sensor II after contacting with the target gas was significant. Therefore, Sensor II whose metal layer was doped with conjugated molecules had a better sensitivity (sensing performance) to toluene gas than Sensor C whose metal layer was not doped with conjugated molecules.

### Example 4

Measurement of Gas Selectivity of Gas Sensor

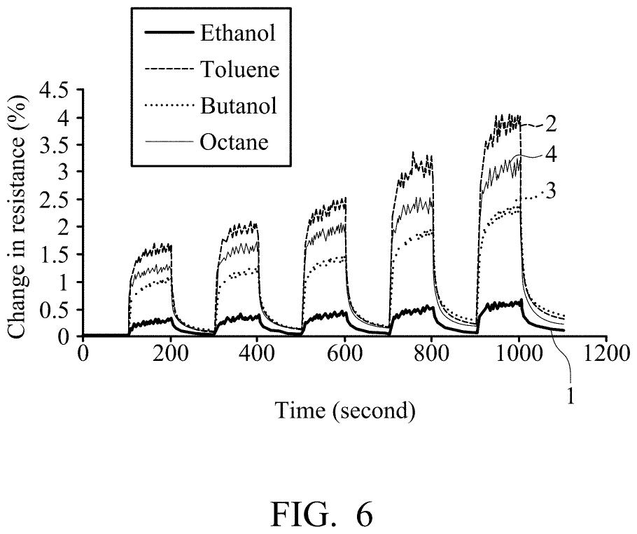

Example 4 illustrates the influence of the metal layers doped with conjugated molecules in the gas sensors on the gas selectivity of the gas sensor. First, Sensor III was provided. In Example 4, the metal layer in the gas sensor described above was mainly formed of nano-gold particles with octyl groups attached to the surface. The metal layer of Sensor III was doped with conjugated molecules

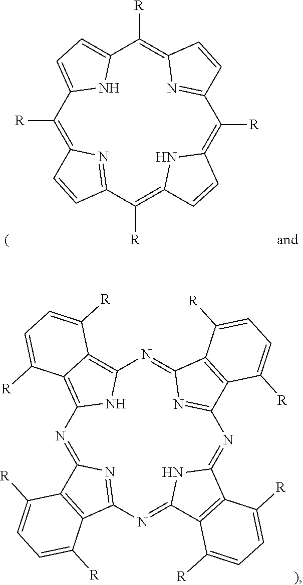

where R was —O—(CH₂)₃CH₃. The doping concentration ratio was 1:2,000. Target gases such as ethanol, toluene, butanol, and octane with a concentration from 400 ppm to 1,000 ppm were introduced into the gas sensor and the gas selectivity of the gas sensor was measured. 400, 500, 600, 800, and 1,000 ppm of ethanol, toluene, butanol, and octane gases were introduced at 100, 300, 500, 700, and 900 seconds, respectively. The measurement results are shown in FIG. 6.

In FIG. 6, Curve 1 shows the change in the resistance of Sensor III after contacting with ethanol gas. Curve 2 shows the change in the resistance of Sensor III after contacting with toluene gas. Curve 3 shows the change in the resistance of Sensor III after contacting with butanol gas. Curve 4 shows the change in the resistance of Sensor III after contacting with octane gas. As shown in FIG. 6, no matter what kinds of target gases were introduced and no matter what time the target gases were introduced, the change in the resistance of Sensor III after contacting with different target gases was significantly distinct. Therefore, Sensor III whose metal layer was doped with conjugated molecules exhibited a high degree of selectivity to various target gases. In other words, different types of target gases with different concentrations can be detected by the gas sensor.

The foregoing has outlined features of several embodiments so that those skilled in the art may better understand the detailed description that follows. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A gas sensor, comprising:
a substrate;
a plurality of electrodes formed on the substrate; and
a metal layer formed on the substrate and the electrodes, wherein the metal layer comprises a plurality of first molecules doped with a plurality of second molecules, wherein each of the first molecules comprises a metal particle and a plurality of carbon chains connected to a surface of the metal particle, and each of the second molecules comprises a conjugated structure.

2. The gas sensor as claimed in claim 1, wherein the metal particle in the first molecules comprises Au, Ag, Cu, Sn, Pd, Pt, Ni, Co, or Al.

3. The gas sensor as claimed in claim 1, wherein a number of carbon atoms in the carbon chains in the first molecules are between 6 and 24.

4. The gas sensor as claimed in claim 1, wherein the carbon chains in the first molecules are connected to the surface of the metal particle through an anchor unit.

5. The gas sensor as claimed in claim 4, wherein the anchor unit comprises an S atom, a P atom, or an N atom.

6. The gas sensor as claimed in claim 1, wherein the second molecules comprise a nitrogen-containing cyclic conjugated structure, a sulfur-containing cyclic conjugated structure, or a cyclic conjugated structure with double bonds.

7. The gas sensor as claimed in claim 1, wherein the second molecules comprise a nitrogen-containing cyclic conjugated structure modified by functional groups, a sulfur-containing cyclic conjugated structure modified by functional groups, or a cyclic conjugated structure with double bonds modified by functional groups.

8. The gas sensor as claimed in claim 7, wherein the functional groups comprise heterocyclic compounds.

9. The gas sensor as claimed in claim 1, distances between the first molecules are increased by the conjugated structure of the second molecules.

10. The gas sensor as claimed in claim 1, wherein a doping concentration ratio of the second molecules to the first molecules is between 1:2 and 1:100,000.

11. The gas sensor as claimed in claim 1, wherein the doping concentration ratio of the second molecules to the first molecules is between 1:20 and 1:10,000.

12. The gas sensor as claimed in claim 1, wherein the first molecules are physically mixed with the second molecules.

13. The gas sensor as claimed in claim 7, wherein the first molecules form covalent bonds with the second molecules.

14. The gas sensor as claimed in claim 13, wherein the metal particle in the first molecules forms covalent bonds with the functional groups in the second molecules.

15. The gas sensor as claimed in claim 1, wherein target gases detectable by the gas sensor comprise volatile organic compounds gases.

16. The gas sensor as claimed in claim 1, wherein target gases detectable by the gas sensor comprise amine gases, nitrogen oxide gases, or explosive gases.
